(19) United States
(12) Patent Application Publication
(10) Pub. No.: US 2005/0019390 A1
(43) Pub. Date: Jan. 27, 2005

(54) Dosage system and dosage vehicle therefor

(76) Inventor: John Kohnke, NSW (AU)

Correspondence Address: KNOBBE MARTENS OLSON & BEAR LLP, 2040 MAIN STREET, FOURTEENTH FLOOR, IRVINE, CA, 92614, US

(21) Appl. No.: 10/493,741

(22) PCT Filed: Oct. 25, 2002
(86) PCT No.: PCT/AU02/01448

Related U.S. Application Data
(60) Provisional application No. 60/339,257, filed on Oct. 26, 2001.

(30) Foreign Application Priority Data
Oct. 26, 2001 (AU) PR 8491
Oct. 30, 2001 (AU) PR 8580

(51) Int. Cl. A61K 9/20; D04H 1/00

(52) U.S. Cl. 424/464; 264/109

(57) ABSTRACT

A dosage system for the delivery quantities and selections of a therapeutically or nutritionally effective substance comprising individual dosage forms, including pellets, prills, beadlets, beads, granules, tablets or caplets containing nutritional and therapeutic substances that are separated to reduce inter-reaction between the compounds that could or can occur when they are mixed together in the one formulation. The individual dosage forms are blended in a selected formulated ratio, to provide a targeted therapeutic dose or food supplement relative to the specific need of the animal or human to deliver medication or correct low or inadequate nutrient levels in the diet.

The present invention relates to a dosage system and a dosage vehicle therefor and, more particularly, to a vehicle and system particularly, although not exclusively, adapted to the delivery of therapeutically effective and/or nutritionally effective substances by oral ingestion for animals.

### BACKGROUND

It is known, for example in the case of humans, to provide vitamin supplements in tablet form.

However, when it comes to providing supplements to animals, particularly for example large animals such as horses, the sheer volume of supplement required to be effective teaches away from a simple pill-based approach.

In addition vitamin supplements have been constrained in the way they can be combined to provide targeted treatments.

It is an object of the present invention to address or ameliorate one or more of the abovementioned disadvantages.

### BRIEF DESCRIPTION OF INVENTION

Accordingly, in one broad form of the invention there is provided a dosage system for delivery of predetermined quantities of predetermined selections of therapeutically or nutritionally effective substance over predetermined time intervals into the body of animal; said dosage system comprising:

(a) At a first location sorting a dosage vehicle according to an at least one primary characteristic;

(b) At said first location selecting and fixing at a first mixing time one or more of said dosage vehicles in accordance with a predetermined selection and predetermined quantity thereby to obtain a first dosage of a predetermined selection and predetermined quantity of said therapeutically or nutritionally effective substance specifically adapted for a predetermined target animal or target animal group;

(c) Transporting said first dosage to a second location;

(d) Selecting and mixing at a second mixing time said first dosage with a predetermined selection and predetermined quantity of feed material thereby to obtain a first feed mix.

(e) administering said first feed mix to said target animal or target animal group at a first predetermined administration time which is at or around said second mixing time;

(f) repeating steps (d) and (a) at predetermined repeat time intervals thereby to maintain a predetermined therapy profile in said target animal or target animal group over a predetermined therapy period.

Preferably said system further includes attributing at least one sub-characteristic to an at least one dosage vehicle.

Preferably said at least one sub-characteristic is such that it can be utilized to distinguish one dosage vehicle from another on the basis of said at least one primary characteristic.

Preferably said dosage vehicle is sorted according to said at least one sub-characteristic.

Preferably said system further includes a second sub-characteristic.

Preferably said second sub-characteristic is selected from odour, density, size, volume, diameter, length.

Preferably said predetermined repeat time interval is 1 day.

Preferably said predetermined repeat time interval is 0.5 day.

Preferably said predetermined therapy period is at least six months.

Preferably said predetermined therapy period is approximately 3 months.

Preferably said predetermined therapy period is approximately 2 months.

Preferably said predetermined therapy period is approximately 1 month.

Preferably a second dosage is administered in a subsequent predetermined therapy period, thereby to tailor therapy for said animal as a function of time.

Preferably said dosage vehicle is in the form of a predetermined quantity of a topical substance.

Preferably said dosage vehicle is in the form of a predetermined quantity of a solid, ingestible substance.

Preferably said solid, ingestible dosage is in the form of a pellet, prill, beadlet, bead, granule, tablet, caplet or flake.

Preferably said predetermined target animal is selected from a dog, a pig, a cow or a fish.

Preferably said predetermined target animal is a horse.

Preferably said predetermined target animal is a human.

In a further broad form of the invention there is provided a method for delivery of predetermined quantities of predetermined selections of a therapeutically or nutritionally effective substance over predetermined time intervals into the body of an animal; said method comprising:

(a) At a first location sorting a dosage vehicle according to an at least one primary characteristic;

(b) At said first location selecting and mixing at a first mixing time one or more of said dosage vehicles in accordance with a predetermined selection and predetermined quantity thereby to obtain a first dosage of a predetermined selection and predetermined quantity of said therapeutically or nutritionally effective substance specifically adapted for a predetermined target animal or target animal group;

(c) Transporting said first dosage to a second location;

(d) selecting and mixing at a second mixing time said first dosage with a predetermined selection and predetermined quantity of feed material thereby to obtain a first feed mix.

(e) administering said first feed mix to said target animal or target animal group at a first predetermined administration time which is at or around said second mixing time;

(f) repeating steps (d) and (e) at predetermined repeat time intervals thereby to maintain a predetermined therapy profile in said target animal or target animal group over a predetermined therapy period.

Preferably said method further includes attributing at least one sub-characteristic to an at least one dosage vehicle.

Preferably said at least one sub-characteristic is such that it can be utilized to distinguish one dosage vehicle from another on the basis of said at least one primary characteristic.

Preferably said dosage vehicle is sorted according to said at least one sub-characteristic.

Preferably said dosage vehicle includes a second sub-characteristic.

Preferably said second sub-characteristic is selected from odour, density, size, volume, diameter, length.

Preferably said predetermined repeat time interval is 1 day.

Preferably said predetermined repeat time interval is 0.5 day.

Preferably said predetermined therapy period is at least six months.

Preferably said predetermined therapy period is approximately 3 months.

Preferably said predetermined therapy period is approximately 2 months.

Preferably said predetermined therapy period is approximately 1 month.

Preferably a second dosage is administered in a subsequent predetermined therapy period, thereby to tailor therapy for said animal as a function of time.

Preferably said dosage vehicle is in the form of a predetermined quantity of a topical substance.

Preferably said dosage vehicle is in the form of a predetermined quantity of a solid, ingestible substance.

Preferably said solid, ingestible dosage is in the form of a pellet, prill, beadlet, bead, granule, tablet, caplet or flake.

Preferably said predetermined target animal is selected from a dog, a pig, a cow or a fish.

Preferably said predetermined target animal is a horse.

Preferably said predetermined target animal is a human.

In yet a further broad form of the invention there is provided a dosage vehicle for a dosage systems, said dosage vehicle comprising a mass in the form of a mixture of dispersed portions of at least one active component in a substantially non-reactive separator material.

Preferably said mass is a compressed mass.

Preferably said mass is a compressed mass compressed by a cold compressing method.

Preferably said substantially non-reactive separator material includes at least one active component.

Preferably said substantially non-reactive separator material is selected so that its concentration and composition is such that its at least cone active component does not interfere with the uptake or other therapeutic or nutritional effect of said at least one active component in said dispersed portions.

Preferably said substantially non-reactive separator material is Calcium Carbonate.

Preferably said substantially non-reactive separator material is Calcium Phosphate.

Preferably the density of said substantially non-reactive separator material is selected so that dosage vehicles of said dosage system all have substantially the same mass irrespective of the composition of said at least one active component.

Preferably the distribution of said at least one active component within said substantially non-reactive separator material is arranged so as to substantially prevent chemical interaction between mechanically juxtaposed dosage vehicles.

Preferably said dosage vehicle is formed from a cold compression process.

Preferably said at least one active component is selected to provide said vehicle with a primary characteristic pertinent to its therapeutic or nutritional effect.

Preferably the composition of said active component and said substantially non-reactive separator material is selected so as to impart at least a first sub-characteristic to said dosage vehicle.

Preferably said first sub-characteristic is a visual characteristic.

Preferably said at least a first sub-characteristic permits a user to distinguish one dosage vehicle from another in accordance with the primary characteristic of each dosage vehicle.

In yet a further broad form of the invention there is provided a dosage of therapeutically or nutritionally effective substance comprising a predetermined selection and predetermined quantity of one or more of the above dosage vehicles.

In yet a further broad form of the invention there is provided a treatment regime for a target animal comprising administering the dosage at predetermined repeat time intervals thereby to maintain a predetermined therapy profile in said animal over a predetermined therapy period.

Preferably said predetermined repeat time intended is 1 day.

Preferably said predetermined repeat time intended is 0.5 day.

Preferably said predetermined therapy period is at least six months.

Preferably said predetermined therapy period is approximately 3 months.

Preferably said predetermined period is approximately 2 months.

Preferably said predetermined Period is approximately 1 month.

Preferably a second dosage is administered in a subsequent predetermined therapy period, thereby to target said animal as a function of time.

Preferably said dosage vehicle is in the form of a predetermined quantity of a topical substance.

Preferably said dosage vehicle is in the form of a predetermined quantity of a solid, ingestible substance.

Preferably said solid, ingestible dosage is in the form of a pellet, prill, beadlet, bead, granule, tablet, caplet or flake.

Preferably said predetermined target animal is a horse.

Preferably said predetermined target animal is a dog, a pig, a cow or a fish.

Preferably said predetermined target animal is a human.

### BRIEF DESCRIPTION OF DRAWINGS

Embodiments of the present invention will now be described with reference to the accompanying drawings wherein:

FIG. 1 is a block diagram of a dosage system in accordance with a first preferred embodiment of the present invention;

FIG. 2 is a diagrammatic representation of dosage vehicles within separate groupings suitable for use with the system of FIG. 1;

FIG. 3 is a diagrammatic representation of an individual dosage vehicle in accordance with an embodiment of the invention;

FIG. 4 is an exemplary arrangement of a first dosage in accordance with an embodiment of the invention;

FIG. 5 is an exemplary embodiment of a second dosage in accordance with an embodiment of the invention;

FIG. 6 diagrammatically illustrates a treatment regime in accordance with an embodiment of the invention.

### DETAILED DESCRIPTION OF PREFERRED EMBODIMENTS

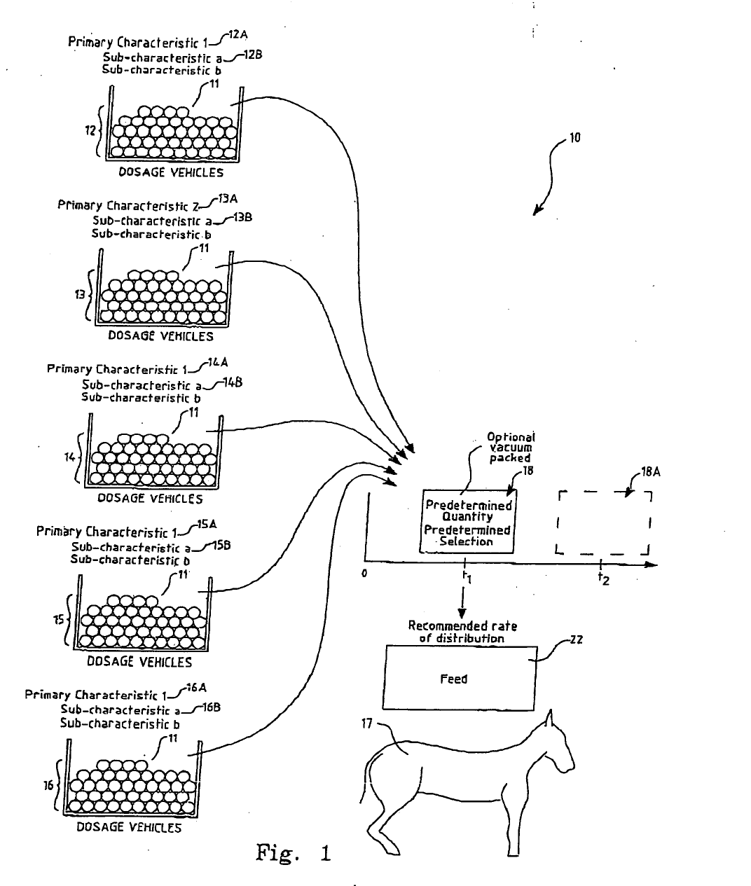

Initially, with reference to FIG. 1, there is illustrated a dosage system 10 in accordance with a first preferred embodiment of the present invention.

In this instance the dosage system 10 comprises five separate groupings of dosage vehicles 11.

In this instance each dosage vehicle of each grouping is comprised of a specific therapeutically effective substance or combination of substances.

In this instance each dosage vehicle 11 is in the form of a compressed cylindrical food pellet termed a “pellet”.

In this specification the term “pellet” refers to distinctive small compressed cylindrical food pellets, typically measuring within the range of 8-12 mm in length and 2-3.5 mm in diameter, formed in a cold-pressed pelleting machine. This method reduces heat damage to heat sensitive nutrients, such as vitamins. Heat damage is a problem in the steam pelleting process used to produce horse feeds.

“Pellets” have been developed to eliminate dust, sift out and food refusal often associated with powdered or granule supplements.

Each pellet is manufactured from high quality ingredients, containing specific nutrients with built-in antioxidants and natural preservative compounds to maintain nutrient potency. They resist crumbling and are formulated to be highly palatable to the target animal.

In this instance there are five grouping wherein first grouping 12 has a first primary characteristic 12A, second grouping 13 has a second primary characteristic 13A, third grouping 14 has third primary characteristic 14A, fourth grouping 15 has a fourth primary characteristic 15A and fifth grouping 16 has fifth primary characteristic 16A as illustrated diagrammatically in FIG. 1.

Each of the primary characteristics 12A, 13A, 14A, 15A, 16A is distinct and separately identifiable one from the other. The primary characteristics define the collective therapeutic effect or other effect of the dosage vehicles 11 making up the respective grouping to which that primary characteristic attaches.

In addition to the primary characteristic each grouping 12, 13, 14, 15, 16 has associated with it at least one sub-characteristic 12B, 13B, 14B, 15B, 16B respectively (identified sub-characteristic “a” in this instance in FIG. 1).

Each grouping can be further identified by further sub-characteristics as appropriate and relevant. In this instance, by way of example, each grouping can be further identified by sub-characteristic “b” in FIG. 1.

In use, for the purpose of therapeutic application to animal 17 a predetermined portion of dosage vehicle 11 from each of the five groupings 12, 13, 14, 15, 16 is mixed together to prepare a dosage 18 comprising a predetermined selection of predetermined quantities of one or more of the groupings 12, 13, 14, 15, 16.

The dosage 18, in this instance, is prepared at the same location at which the individual dosage vehicles 11 are prepared and sorted initially according to sub-characteristic “a”. The dosage 18 is then transported to a separate location at or near where administration is to take place.

At a time of first administration t1 the dosage 18 is mixed into feed 22 and fed to animal 17 resulting in the ingestion of dosage 18.

A similar procedure can be followed to prepare a second dosage 18A for administration at time t2 and so on with a view to maintaining a predetermined therapy profile in the target animal 17 over a predetermined therapy period.

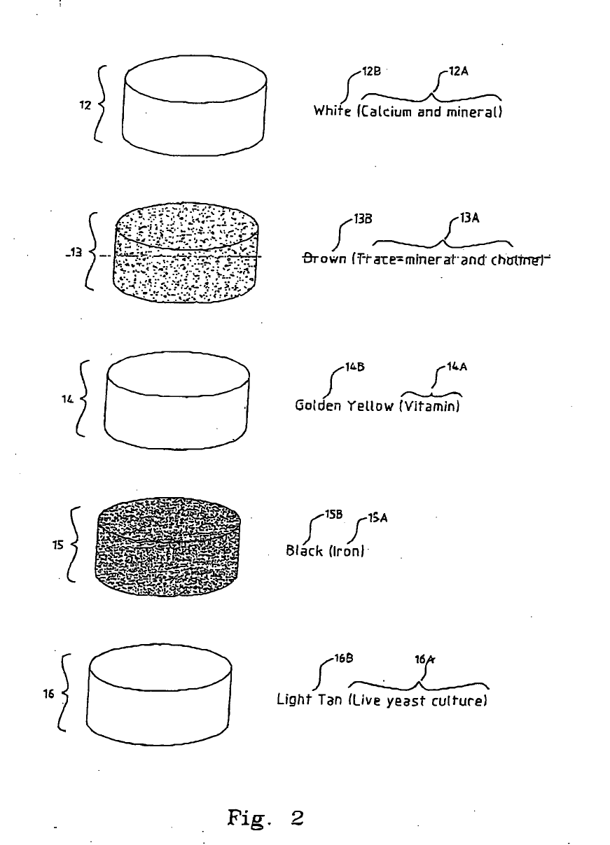

FIG. 2 illustrates particular examples of sub-characteristic “a”suited to link the respective groupings to their primary characteristics 12A, 13A, 14A, 15A, 16A. In this instance the sub-characteristic “a” is based on colour thereby allowing a link to be made between the colour of the dosage vehicles 11 in a particular grouping with the primary (therapeutic) characteristic of that same grouping. So, for example, as illustrated in FIG. 2, first grouping 12 can be made up of pellets of white colour, the pellets containing calcium and mineral supplements as will be described by way of further example below.

Second grouping 13 can have sub-characteristic “a” defined as colour brown with the composition of the pellets comprising trace-minerals and choline.

Similarly third grouping can have a sub-characteristic “a” comprising a golden yellow colour which is associated with a vitamin composition. Fourth grouping 15 can have a sub-characteristic comprising a black colour signifying iron supplement composition. Finally, in this instance, fifth grouping 16 can be identified by a sub-characteristic comprising a light tan colour signifying a pellet composition comprising live yeast culture.

Typically each grouping will contain a chemical composition which has a reasonably long shelf-life. In many instances, however, on associating or mixing dosage vehicles from different groupings the shelf-life, by virtue of the close mechanical association may in the prior art become reduced.

In the instance of the present invention, each dosage vehicle 11, in this instance in the form of a pellet 19 includes an array of active components 20 distributed throughout the pellet 19 within a substantially non-reactive separator material 21. Typically the separator material 21 can be a calcium based material. This arrangement ensures that pellets or dosage vehicles from different groupings and having different primary characteristics, even if closely mechanically associated will not allow the active components 21 to come into such close contact that unwanted reactions will take place prior to ingestion or administration.

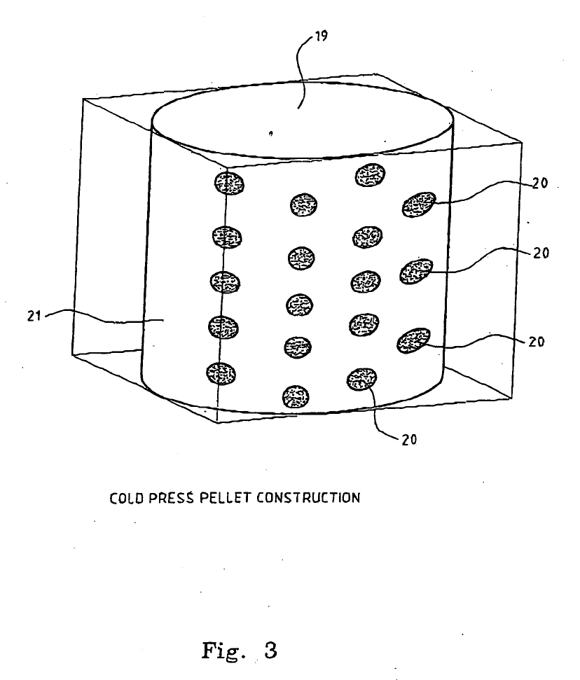

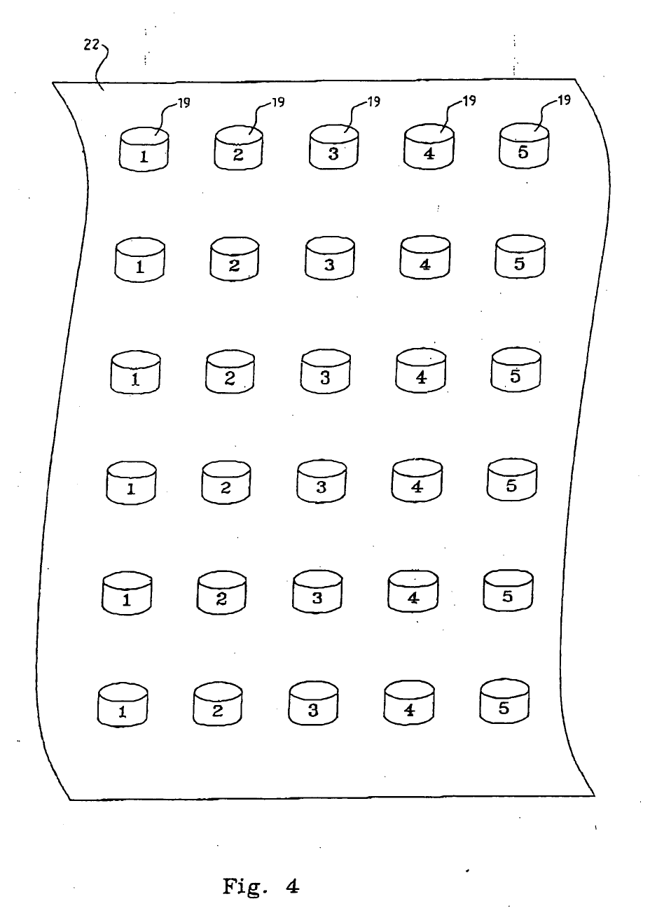

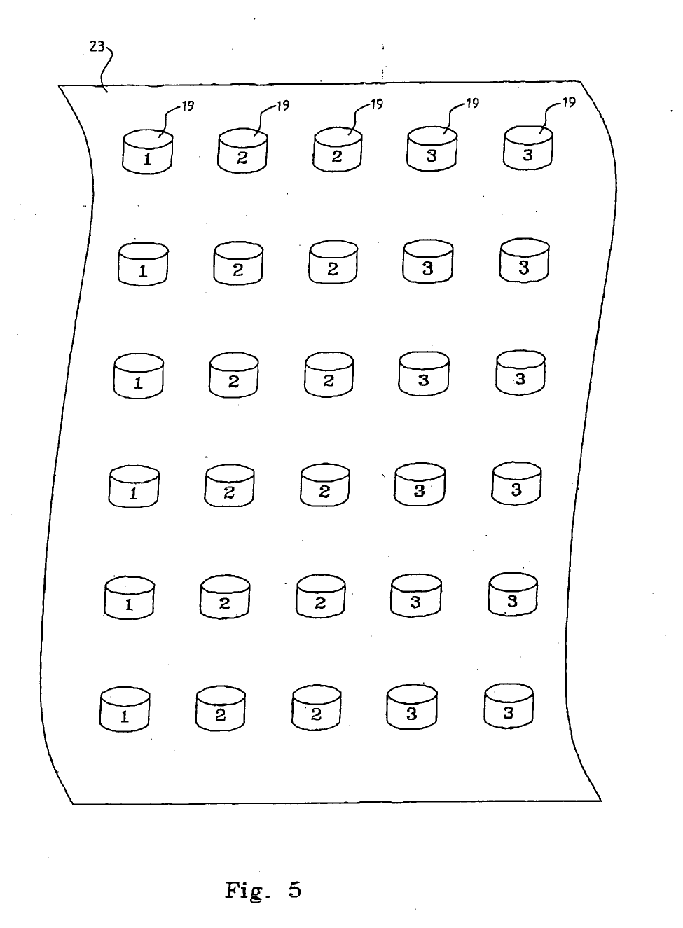

With reference to FIGS. 4 and 5 the particular characteristics of the pellet construction described with reference to FIG. 3 allows pellets/dosage vehicles having different primary characteristics to be aggregated into close mechanical association. So, for example, FIG. 4 allows a dosage 22 to be formulated by aggregating equal portions of dosage vehicles 11 having five different privacy characteristics, namely 20% of type 1 characteristic, 20% of type 2 characteristic, 20%. of type 3 characteristic, 20% of type 4 characteristic and 20% of type 5 characteristic.

In addition the non-reactive separator material 21 in each pellet 19 is selected so that the weight of each pellet, irrespective of the active components 20 within each pellet is approximately the same for each pellet, irrespective of its primary characteristic 12A, 13A, 14A, 15A, 16A. A potential benefit of this characteristic is that the distribution of pellets 19 throughout dosage 22 does not vary over time.

FIG. 5 illustrates an alternative exemplary dosage 23 comprised, in this instance, of 20% of pellets 19 having a type 1 characteristic, 40% of pellets 19 halving a type 2 characteristic an 40% of pellets saving a type 3 characteristic.

It will be understood that different dosages such as dosage 23 and dosage 22 can be made up for targeting specific animals and taking into account some specific characteristics of the animal such as, for example, age, its daily work schedule at that time and so on. Hence dosage 22 may be suitable for a given animal at one time in its life and under certain working conditions whilst dosage 23 might be more suited to that animal at a different time in its life or when the animal is working under different working conditions.

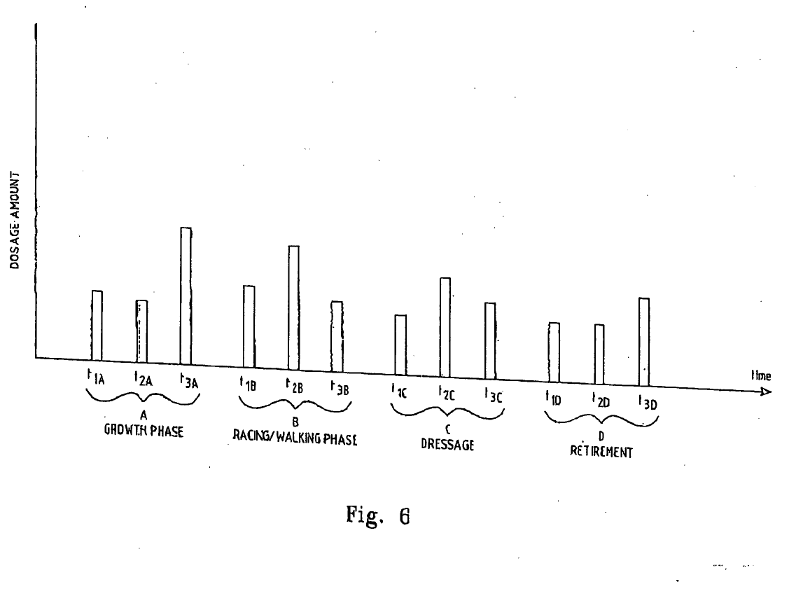

With reference to FIG. 6 there is illustrated in graphical form a “whole of life” treatment regime to which the dosage system 10 of the present invention is suited. In this specific example the animal is a horse passing through a growth phase A, a racing/working phase B, a dressage phase C and a retirement phase D. In growth phase A the administration times T1, T2, T3 are specific to growth phase A and are hence denoted T1A, T2A, T3A (refer FIG. 1). In growth phase A it may be that relevant proportions suited for that particular animal at that particular phase are best provided by dosage 22 of FIG. 4 whilst during the racing/working phase B the dosage 23 of FIG. 5 would be more appropriate administered at time intervals T1B, T2B, T3B.

Thus it will be observed that a highly targeted and animal specific treatment regime can be formulated via dosage system 10, but provided in a cost effective way with the initial production of pellets 19 or like dosage vehicles 11 being performed in the appropriate manufacturing context and followed by the segregation of the various groupings 12, 13, 14, 15, 16 (refer FIG. 1) on the basis of sub-characteristics which are a function of the primary characteristics of the different dosage vehicles 11 being utilized to prepare targeted dosages 18 also at the manufacturing complex following which the dosages 18 can be delivered to the location of the target animal and administered in the manner generally described with reference to FIG. 1 and FIG. 6.

There will now follow specific examples of compositions and treatment regimes suited for application by way of the dosage system 10 as described above. The examples are directed at the instance where the target animal 17 is a horse. However, it will be understood that other target animals can be identified.

### EXAMPLE 1

### Target Animal Group: For Racing, Performance, Equestrian, Breeding and Growing Horses

### Concentrated Trace-Mineral and Vitamin Supplement for Horses

The regime of this example represents a new concept in providing a supplement of trace-minerals and vitamins to make-up shortfalls in the common grain, chaff and hay rations fed to horses in training. The regime is formulated from two separate dosage vehicles blended in a 50:50 ratio to deliver a comprehensive range of nutrients to correct low or inadequate levels in the diet.

### First Dosage Vehicle

### Brown Pellet: The Major Trace-Minerals

Provide all the essential trace-mineral to the latest supplementary levels recommended for racing and performance horses. An adequate intake of these nutrients is important to ensure optimum metabolic function, bone and tissue strength.

These trace-mineral pellets are formulated to maximise the uptake of the individual trace-minerals by a combination of “chelated” (protein complexed) organic forms and inorganic soluble sources of zinc, manganese, copper, iron and cobalt, as wall as iodine, complemented by organic selenium and chromium in yeast complexes. In fact, Cell-Vital™ contains three sources of iron to help ensure optimum uptake and utilisation of this important trace-mineral in the diet of horses in training.

### Second Dosage Vehicle

### Golden Yellow Pellot: The Major Vitamins

Contain a comprehensive range of all vitamins to avoid interaction with trace-minerals that can occur in “all-in one” powders and other bulky pelleted supplements.

The dosage vehicle of this example provide optimum levels of vitamin A to offset losses of this important vitamin following harvest and storage of feed, as well as Vitamin E in its most stable form. A full range of B group vitamins, including assured levels of those most commonly destroyed in feeds, helps to ensure optimum levels necessary for metabolic function. A supplementary level of choline, recognised as a B group vitamin, is included in the trace-mineral pellet formulation, because choline is well-known for its destructive effects leading to a lose of vitamin potency when mixed into an “all-in-one” powdered or liquid vitamin supplement.

The two dosage vehicles of this example provide a high potency, palatable trace-mineral and vitamin supplement formulated to correct low or inadequate levels in the common feeds fed to racing and other horses, thereby effectively meeting the increased needs of the equine athlete.

### Active Ingredients Per Dose

Each 40 g daily dose of a 50/50 blend of the two dosage vehicles provides the following nutrients:

Essential Trace-Minerals

(as proteinate (chelated) and inorganic forms for optimum bioactivity

| | |
|---|---|
| Zinc (Zn) | 350 mg |
| Manganese (Mn) | 350 mg |
| Copper (Cu) | 160 mg |
| Iron (Fe) | 350 mg |
| Cobalt (Co) | 1.2 mg |
| Iodine (I) | 2.3 mg |
| Selenium (as Selenium yeast Sel-Plex-50 ™*) | 1.4 mg |
| Chromium (as chromium yeast Biochrome ™*) | 5 mg |

Vitamin Co-factors

| | |
|---|---|
| Vitamin A | 50,000 iu (15 mg) |
| Vitamin D3 | 5,000 iu (125 μg) |
| Vitamin E | 500 iu (500 mg) |
| Vitamin K3 | 20 mg |
| Vitamin B1 | 40 mg |
| Vitamin B2 | 50 mg |
| Niacin (Vitamin B3) | 130 mg |
| Pantothenate (Vitamin B5) | 50 mg |
| Vitamin B6 | 20 mg |
| Vitamin B12 | 100 μg |
| Folic acid | 15 mg |
| Biotin | 250 μg |
| Choline (in trace-mineral supplet ™) | 300 mg |

Each 40 g of Cell-Vital™ also contains

| | |
|---|---|
| Calcium (Ca) | 3.6 g |
| Phosphorus (P) | 1.2 g |

*Trademark of Alltech Biotechnology Inc, Kentucky USA

Because the two dosage vehicles contain nutrients in a concentrated form to make-up shortfalls in the ration, it is best to divide the full daily amount above 20 g between the morning and evening feed to help ensure optimum uptake or nutrients, avoiding overloading of the absorption mechanism in the digestive tract.

The 50:50 blend of trace-mineral and vitamin pellets are highly palatable and well accepted by horses, even those considered to be suspicious eaters.

The daily dose ranges from 20 g a day for horses in light work to 30 g daily (as 2×15 g doses) for horses in pre-training, to 40 g daily (2×20 g doses) for horses that are in full race or upper level equestrian training

Available in: 3.0 kg plastic bucket (75×40 g doses)

15 kg plastic bucket (375×40 g doses)

### EXAMPLE 2

### Target Animal Grouping: For Racing & Performance Horses Concentrated Mineral, Trace-Mineral and Vitamin Supplement For Racing and Performance Horses

The dosage is formulated as a high potency “all-in-one” supplement to ensure that the demand for both major and micronutrients, which may be inadequate in the diet of horses in hard training and group level racing and upper level athletic performance, is satisfied.

The dosage of this example is composed of five (5) separate types of pallets, each providing specific nutrients in a stable cold-pressed form to make up shortfalls in the grain, chaff and hay diets to me)et the elevated metabolic activity associated with hard, fast or prolonged exercise.

### First Dosage Vehicle

### White Dosage Vehicles: The Major Minerals

Contain calcium, phosphorus and magnesium, with Vitamin A and D, as the major skeletal struotural minerals to help correct imbalanced and low levels in grain based diets, thus ensuring the maintenance of a strong musculo-skeletal system.

### Second Dosage Vehicle

### Brown Dosage Vehicle: The Major Trace-Minerals

Provide all the essential trace-minerals to correct low levels in racing diets, that could otherwise result in reduced musculo-skeletal strength and less than optimum supply of available trace-minerals including zinc, manganese, iron, iodine and copper essential for efficient metabolic function. Both selenium and chromium are included as bioactive organic bioplexes with yeast, which helps ensure optimum utilisation and tissue cell bioactivity.

### Third Dosage Vehicle

### Golden Yellow Dosage Vehicles: The Major Vitamins

These fortify the diet with a comprehensive range of vitamins to correct low levels and loss of potency in feed during storage. Adequate levels of these important micronutrients are essential for metabolism, energy utilisation, liver function, the maintenance of the appetite and well-being of horses subjected to hard training and repeated upper level racing and competition.

### Fourth Dosage Vehicle

### Black Dosage Vehicles: Three Forms of Iron

Deliver an additional source of iron in 3 forms, with vitamin C, Vitamin B12 and folic acid, which in conjunction with the trace-minerals and vitamins contained in the other pellets, help to correct inadequate dietary levels to meet the need for this important trace-mineral for optimum tissue enzyme and oxygen transport function in the blood and muscles.

### Fifth Dosage Vehicle

### Red Dosage Vehicles: Live Yeast Culture and Vitamin E

These pellets complete the high potency nutritional package supplied by the regime of this example.

Based on a live yeast culture which is known to provide additional micronutrients and assist release of nutrients from grains and hay, these pellets also contain additional Vitamin E to meet upper level demands, as well as Vitamin C for stabled horses without access to pasture or green feed.

The regime of this example eliminates the need for separate supplements of calcium, iron, Vitamin E and other vitamins that substantially increase the costs of feeling. Its scientifically formulated cold-pressed pellets help ensure optimum nutrient potency even in this “all-in-one” supplement to the last dose in the container.

Even the way that this regime is dosed is different. Because of its high potency, it is recommended that it be provided as two half doses, one in the morning feed and the other in the evening feed, to ensure optimum uptake from the digestive system.

Where the regime of Example 1 is used as a routine supplement, the regime of Example 2 may be substituted for 2 days prior to, and for 2 days after, racing or hard competition.

The regime of Example 2 is formulated as a comprehensive mineral, trace-mineral and vitamin supplement with a bioactive live yeast culture to help correct dietary shortfalls thereby maintaining optimum metabolic and digestive function in upper level equine athletes. However, because demand can change relative to the diet, additional supplementation of calcium and phosphorus, as well as a salt mix, may be required to ensure adequate levels of these important nutrients.

### Active Ingredients Per Dosage

Each 120 g of dosage of Example 2, the standard recommended dose, divided between morning and afternoon feed (2×60 g doses each day), provides the following comprehensive range of nutrients in 5 separate pellets:

Major Minerals and Digestive Nutrients

| | |
|---|---|
| Calcium (Ca) | 11.8 g |
| Phosphorus (P) | 4.1 g |
| Magnesium (Mg) | 2.8 g |
| Live Cultured Yeast | 10.0 g |
| (as yea-Sacc1026 ™) | |

Essential Trace-Minerals

(as proteinate (chelated) and inorganic forms for optimum bioactivity)

| | |
|---|---|
| Zinc (Zn) | 357 mg |
| Manganese (Mn) | 357 mg |
| Copper (Cu) | 163 mg |
| Iron (Fe) | 477 mg |
| Cobalt (Co) | 1.2 mg |
| Iodine (I) | 2.35 mg |
| Selenium (as Selenium yeast Sel-Plex-50 ™*) | 1.4 mg |
| Chromium (as chromium yeast Biochrome ™*) | 5.1 mg |

Vitamin Co-factors

| | |
|---|---|
| Vitamin A (Retinol) | 56,130 iu (16.84 mg) |
| Vitamin D3 (Cholecalciferol) | 5,613 iu (140.3 μg) |
| Vitamin E (d1-α-tocopherol) | 1285 iu (1285 mg) |
| Vitamin K3 (Menadione) | 20.4 mg |
| Vitamin B1 (Thiamine) | 40.8 mg |
| Vitamin B2 (Riboflavin) | 51 mg |
| Niacin (Vitamin B3) | 132.6 mg |
| Pantothenate (Vitamin B5) | 51 mg |
| Vitamin B6 (Pyridoxine) | 40.8 mg |
| Vitamin B12 (Cyanocobalamin) | 222 μg |
| Folic acid | 19.8 mg |
| Vitamin H (Biotin) | 252 μg |
| Choline | 300 mg |
| (contained in trace-mineral supplet ™ as | |
| choline chloride to avoid vitamin | |
| destruction during storage) | |
| Vitamin C (as sodium and | 1.03 g |
| calcium ascorbate for optimum stability | |
| and bioactivity) | |

*Trademark of Alltech Biotechnology Inc, Kentucky U.S.A.

The pellets of Example 2 are formulated as 4 major supplements to correct dietary imbalances and inadequacies of calcium and phosphorus and magnesium, thereby helping to maintain musculoskeletal structure and metabolic activity; additional iron and Vitamin C for blood maintenance and metabolic function: Vitamin E for metabolic efficiency, as well as a comprehensive range of trace-minerals end vitamins, including a live yeast culture, to meet the elevated needs of horses in hard training on grain and hay base diets.

A daily dose of the dosage of Example 2, by combining 4 major supplement groups in an innovative form to avoid nutrient interactions, saves both tine and money compared to mixing 4 separate supplements into a feed.

Remember, This example is a highly concentrated nutritional supplement which should be given as two half doses per day to avoid overload of gut absorption sites of calcium, trace-minerals and vitamins. Additional calcium and phosphorus may be required in horses fed on high grain or high lucerne diets to counteract imbalanced intakes in these feeds. A supplement of salts should be provided relative to the work effort and duration, and climatic conditions.

The daily dose ranges from 100 g (2×50 g doses) for horses weighing 400-475 kg, and 120 g (3×60 g doses) for horses weighing 475 to 550 kg in hard race training or upper level equestrian competition, including polo horses, eventing, endurance and showjumping horses.

Available in: 3.0 kg plastic buckets (25×120 g doses)

15 kg plastic buckets (125×120 g doses)

### EXAMPLE 3

### Target Animal Group: For Growing and Breeding Horses Mineral, Trace-Mineral and Vitamin Supplement for Growing and Breeding Horses

A comprehensive supplement containing three (3) separate dosage vehicles or pellets, combined in a scientifically blended ratio that meets the specific needs of growing and breeding horses.

### First Dosage Vehicle

### White Dosage Vehicle: The Major Minerals

Contain calcium, phosphorus and magnesium, with Vitamin A and D to correct low, imbalanced or inadequate levels in diets, thereby helping to ensure optimum bone formation and development. The ratio of calcium to phosphorus at 2.7 calcium to 1.0 phosphorus is specially matched to meet critical mineral balance on both grass and logume based pastures, as well as diets supplemented with lucerne or meadow hay.

This combination ensures adequate phosphorus is available to maintain fertility in breeding mares, as well as the skeletal foundation of unborn foals in late-term pregnant mares and avoid bone mineral imbalances and deficiencies in the diets of growing horses.

### Second Dosage Vehicle

### Brown Dosage Vehicle: The Major Trace-Minerals

This innovative vehicle in the form of a pellet contains all the essential trace-minerals to make up dietary shortfalls, thereby helping to ensure sound bone and joint development—focusing on targeted supplementation with the three trace-minerals most likely to be inadequate or imbalanced in the diet relative to the needs of growing horses—zinc, manganese and copper,—as well as iodine and selenium, for growth and assured fertility in breeding horses.

This pellet provides a combination of ‘chelated’ or protein complexed and soluble elemental trace-minerals to ensure optimum uptake and utilisation, Ed thereby assists in reducing the risk of bone and joint abnormalities in growing horses and unborn, late-term foals.

### Third Dosage Vehicle

### Golden Yellow Dosage Vehicle: The Major Vitamins

A full range of vitamins is compounded into a separate pellet to ensure that optimum stability of the individual vitamins is maintained by eliminating direct contact with destructive trace-minerals.

The vitamin content helps to supplement the natural losses of vitamins in feeds during storage and processing, assuring optimum activity of the mineral and trace-mineral content where vitamin co-factors, such as Vitamin A, D, E and many B group vitamins, are vital for structural and metabolic function.

This regime is an innovative supplement that overcomes the problems of sift-out and powder loss from dry feeds fed out to paddocked horses.

### Active Ingredients Per Dose

The dose rates of this example are relative to the ration blend and the specific needs related to the age and growth rate of foals, weanlings and yearlings, and the stage of pregnancy and lactation in broodmares. Dose rates are recommended on the expected mature weight that will be achieved in a growing horse, and the actual body weight of a mare or breeding stallion. For this reason, the dose rate ranges from 60-150 g daily, with the average dose rate being 105 g daily.

| | 105 grams | 150 grams |
|---|---|---|
| | (weanlings 6 mths | Early lactation |
| Major Minerals | mature to 500 kg) | mere 500 kg |
| Calcium (Ca) | 11.8 g | 16.9 g |
| Phosphorus (P) | 4.1 g | 5.9 g |
| Magnesium (Mg) | 3.9 g | 5.6 g |

Essential Trace-Minerals

(as proteinate (chelated) and inorganic forms for optimum bioactivity)

| | | |
|---|---|---|
| Zinc (Zn) | 312 mg | 446 mg |
| Manganese (Mn) | 312 mg | 446 mg |
| Copper (Cu) | 143 mg | 204.3 mg |
| Iron (Fe) | 312 mg | 446 mg |
| Cobalt (Co) | 1.07 mg | 1.53 mg |
| Iodine (I) | 2.05 mg | 2.93 mg |
| Selenium (as Selenium yeast Sel-Plex-50 ™*) | 1.25 mg | 1.78 mg |
| Chromium | 4.5 mg | 6.4 mg |
| Choline (as Chromium yeast Biochrome ™) | 263 mg | 375 mg |

Vitamin Co-factors

Vitamin A (Retinol) 41,475iu (12.44 mg) 59,250iu (17.78 mg)

Vitamin D3 (Cholecalciferol) 4,147iu (103 μg) 5,925iu (148 μg)

Vitamin E (dl-α-tocopherol adsorbate) 341iu (341 mg) 487iu

| | | |
|---|---|---|
| Vitamin K3 (Menadione) | 13.65 mg | 19.5 mg |
| Vitamin B1 (Thiamine) | 27.3 mg | 39 mg |
| Vitamin B2 (Riboflavin) | 34.13 mg | 48.76 mg |
| Niacin (Vitamin B3) | 88.73 mg | 126.7 mg |
| Pantothenate (Vitamin B5) | 34.13 mg | 48.76 mg |
| Vitamin B6 (Pyridoxine) | 27.3 mg | 39 mg |
| Vitamin B12 (Cyanocobalamin) | 68.25 μg | 97.5 mg |
| Folic acid | 10.24 mg | 14.6 mg |
| Vitamin H (Biotin) | 168 μg | 240 μg |

*Trademark of Alltech Biotechnology Inc, Kentucky USA

Available in: 3 kg plastic buckets (28×105 g doses)

15 kg plastic buckets (142×105 g doses)

### EXAMPLE 4

### Target Animal Group: For Racing, Performance, Equestrian, Breeding and Growing Horses

Calcium, Phosphorus, Magnesium and Trace-Minerals, with Vitamin A and D, for Horses

This regime is not an ordinary calcium supplement—it is formulated to provide essential bone minerals, complemented by trace-minerals and Vitamin A and D to ensure common dietary inadequacies and imbalances are corrected.

This dosage regime contains two (2) pellets in a blend that helps overcome low or inadequate levels in grain based feeds, or low lucerne diets, fed to racing, performance and stud horses.

### First Dosage Vehicle

### White Dosage Vehicle: The Major Minerals

Contain calcium, phosphorus and magnesium, with Vitamin A and D, to make up shortfalls in the feed, and provide adequate major minerals, especially calcium, thereby helping to maintain musculo-skeletal soundness in horses in hard training.

### Second Dosage Vehicle

### Brown Dosage Vehicle: The Major Trace-Minerals

Provide a scientifically formulated amount of essential trace-minerals, particularly zinc, manganese, copper and selenium, that are low in many home-mixed diets due to widespread soil deficiencies and inherent imbalances in grains and hays.

A daily supplement of this dosage provides additional bone and joint minerals, am well as a wide range of essential trace-minerals shown to be necessary for adaptive bone modeling that strengthens the muscular-skeletal system in response to increased load-bearing when horses are in a training program.

The dosage of this example is complementary to the dosage of Example 2 as a source of calcium and bone minerals to make-up shortfalls in the diets of racing and breeding horses.

This dosage is an innovative way of providing a highly palatable source of calcium and a “top-up” of trace-minerals, which, as they are blended from two separate pellets, help reduces the risk of sift out and separation common with powdered calcium supplements mixed into feeds.

Horses relish the taste of the pellets of this regime as compared to bland calcium powders, further reducing sift-out.

This regime provides an amount of calcium, along with phosphorus, Vitamin A and Vitamin D that facilitates its uptake and blood balance, which can be absorbed efficiently.

This regime does not provide a large quantity of calcium in a single dose as contained in many calcium supplements formulated for high grain diets. This is because 90% of calcium is absorbed from the small intestine and when given once daily in large quantities of insoluble calcium carbonate, much of the calcium is not able to be absorbed and is passed into the hindgut where it is not available, and eventually it passes into the droppings.

It is not the sheer amount of calcium that is supplied that is important, it's the balance between other minerals, including magnesium and phosphorus, and the need to avoid overload of excess beyond the absorption limit, that determines the efficiency of calcium uptake and balance between calcium and phosphorus in the horse's blood and body tissues. For this reason, it is recommended that it is given twice daily, mixed into the feed to help ensure more efficient and complete uptake and utilisation.

This regime is a balanced form, containing a 2.7-1.0 calcium to phosphorus ratio, as compared to 8:1. ratio in cheaper, limestone based supplements that are often also given at higher dosage rates. The inclusion of complementary levels of trace-minerals helps to avoid the risk of over-supply and imbalances that can occur when another supplement containing these nutrients and vitamins is added to the diet.

### Active Ingredients Per Dose

The average dose of this example ranges from 60-120 grams per day, given in equal doses divided between morning and evening feeds.

| | | | 120 g |
|---|---|---|---|
| | 60 g | 90 g | (Racing, |
| | (Light Work) | (Medium Work) | Pre-Training) |
| Calcium (Ca) | 8.9 g | 13.4 g | 17.8 g |
| Phosphorus (P) | 3.3 g | 4.9 g | 6.6 g |
| Magnesium (Mg) | 3.05 g | 4.6 g | 6.1 g |

This regime also contains:

| | | | | | | |
|---|---|---|---|---|---|---|
| Zinc (Zn) | 52.5 | mg | 78.75 | mg | 105 | mg |
| Manganese (Mn) | 52.5 | mg | 78.75 | mg | 105 | mg |
| Copper (Cu) | 24 | mg | 36 | mg | 48 | mg |
| Iron (Fe) | 52.5 | mg | 78.75 | mg | 105 | mg |
| Cobalt (Co) | 0.18 | mg | 0.27 | mg | 0.36 | mg |
| Iodine (I) | 0.35 | mg | 0.52 | mg | 0.69 | mg |
| Selenium (Se) | 210 | μg | 315 | μg | 420 | μg |
| Chromium (Cr) | 0.75 | mg | 1.13 | mg | 1.5 | mg |
| Vitamin A (Retinol) | 5,700 | iu | 8550 | iu | 11,400 | iu |
| Vitamin D3 | 570 | iu | 855 | in | 1140 | iu |
| (Cholecalciferol) | | | | | | |

Available in: 3.5 kg plastic buckets (35-70 doses)

15 kg plastic buckets (150-300 doses)

### EXAMPLE 5

### Target Animal Grouping: For Racing and Performance Horses

### Iron Supplement for Equine Athletes

This example is a new concept in the method of supplementing the trace-mineral iron when requirements in horses are elevated in excess of that naturally contained in, or available from digestion of feed.

The traditional hay and grain diets of horses in training contain from 1,500-1,800 mg of elemental iron in the daily ration. However only 15-17% of this iron is able to be absorbed or is ‘bioavailable’ to the average horse, particularly from dry, mature hays and grains.

The regime of this example is formulated as an innovative supplement containing three (3) forms of iron, with complementary Vitamin C, Vitamin B12 and folic acid, that is formulated to, correct low or inadequate levels in the diet relative to a horse's specific daily requirement.

It is primarily a source of iron only, it is recommended to be used as a booster source of iron during the critical period prior to hard exercise or racing, when more iron is required by the metabolic enzymes and oxygen transport compounds in the muscles during periods of maximal oxygen uptake.

It is formulated for use in conjunction with the regime of Example 1 trace-mineral and vitamin supplement where it can be used to make up dietary shortfalls of iron in the range of essential trace-minerals and vitamins for hard working horses.

The pellets of this example contain three (3) forms of iron:

Iron protein bioplex a bioactive, organic form of iron that is carried on a protein (15% iron-protein chelate) to facilitate its uptake from the small intestine.

Iron carbonyl a source of 98% pure iron in micronised form, which has been (98% iron) shown to be the most available form of supplementary iron for human and animal diets.

Iron sulfate a well absorbed form of iron that provides a reservoir of (23% iron) elemental iron in the gut for more sustained release.

A matched amount of sodium and calcium ascorbate, as stable forms of vitamin C, are included to facilitate the uptake of iron from the small intestine, complemented by Vitamin B12 and folic acid to assist in its utilisation. Whey powder is used to provide a source of phospholipid compounds to help maintain the intestinal lining and minimise irritation by iron and other elemental mineral compounds.

### Active Ingredients Per Dose

Each 20 g of Cell Iron pellets, the mid range dose, provides the following nutrients:

| | 15 grams | 20 grams | 25 grams |
|---|---|---|---|
| Iron (as Proteinate) | 135 mg | 180 mg | 225 mg |
| Iron (as caxbonyl) | 29.4 mg | 39.2 mg | 49 mg |
| Iron (as sulfate) | 75.6 mg | 100.8 mg | 126 mg |
| Total Iron (16 mg/g) | 240 mg | 320 mg | 400 mg |

Also contains per dose:

| | | | |
|---|---|---|---|
| Sodium ascorbate | 120 mg | 160 mg | 200 mg |
| Calcium ascorbate | 120 mg | 160 mg | 200 mg |
| Cyanocobalamin (Vit. B12) | 240 μg | 320 μg | 400 μg |
| Folic acid | 9 mg | 12 mg | 15 mg |
| Whey powder containing | 1500 mg | 2000 mg | 2500 mg |
| 13% crude protein and | | | |
| gut protective phospholipids | | | |

The label dose rates include recommendations for horses in pre-training, where additional iron may be beneficial to correct inadequate levels in the diet when red cell numbers increase in response to regular exercise.

The dose recommendations also take into account other sources of iron provided in the diet, such as by the addition of supplements of Example 1 (350 mg iron/40 g dose) or other supplements, so as to avoid excess amounts of iron that would be not fully absorbed or utilised.

Additional recommendations are provided for horses in advanced and full training in both cool and hot, humid climates. Iron is excreted in the sweat (23 mg iron/Litre of sweat) of horses during hot weather. The basic hay and grain diet is unlikely to either provide this amount for the elevated needs of horses in training or allow the horse to replace daily losses when a horse sweats heavily under hot conditions.

The vehicles of this example comprise cold-pressed pellets, which are dust free, difficult to sift out and do not separate in the feed mix.

The pellet of this example can be mixed into the feed to provide an additional source of iron to supplement low or inadequate levels in the diet, prior to racing and herd exercise, or as a regular 3 day course of iron at 10-14 day intervals for horses in race training on grain and hay based diets.

Available in: 1.2 kg plastic tubs (60×20 g doses)

3.5 kg plastic buckets (175×20 g doses)

### EXAMPLE 6

### Target Animal Group: Show and Pleasure Horses, Ponies and Miniature Horses

### Calcium, Trace-Mineral and Vitamin Supplement for Adult Show and Pleasure Horses, Ponies and Miniature Horses

This supplement formulated for the lightly worked show and pleasure horse that is economical, easy to use and makes up the shortfalls in pasture as well as in a hard feed ration.

This regime provides a comprehensive and high quality range of minerals, trace-minerals and vitamins—but in three (3) separate small, palatable cold-pressed pellets.

### First Dosage Vehicle

### White Colored Pellets: The Major Minerals

Contain calcium, complemented by Vitamin A and D to assist its uptake and regulation, as well as phosphorus and magnesium—the major minerals to offset low levels in chaff and hay diets.

### Second Dosage Vehicle

### Brown Pellets: The Major Trace-Minerals

Provide a wide range of essential trace-minerals, many in protein bioplex (protein chelated) forms to maximise uptake, including selenium and chromium yeasts to meet the needs of light work, thereby providing additional tract-minerals that help ensure optimum coat condition.

### Third Dosage Vehicle

### Golden Yellow Pellets: The Major Vitamins

These pellets deliver a comprehensive package of essential vitamins often lost in dried and stored feeds making up shortfalls, thereby assisting metabolism, vitality and health.

The three separate dosage vehicles of this example help assure stability of the individual classes of nutrients, reducing the risk of inter-reaction, chemical binding and loss of potency of trace-minerals and vitamins curing storage and use. They are palatable and a daily supplement can be given to horses at pasture, if necessary, off the hand without having to provide them with a ‘hard’ feed.

It is an innovative supplement that helps turn ‘feed’ into ‘food’—avoiding sifting out and dust common with powdered supplements.

Available in: 3 kg plastic buckets (60×50 g doses for a 450-500 kg horse)

15 kg plastic buckets (300×50 g doses for a 450-500 kg horses)

The number of doses in each pack for Ponies and Miniatures is relative to each individual animal's bodyweight.

### SUMMARY OF EXAMPLES

In summary, important or significant characteristics of the dosage systems of Examples 1 to 6 may be summarized as:

1. Individual solid dosage forms, including pellets, prills, beadlets, beads, granules, tablets or caplets contain nutritional and therapeutic substances that are separated to reduce inter-reaction between the compounds that could or can occur when they are mixed together in the one formulation, and then pressed or formed into a solid dosage form.

NB. Prills are a small ball-like dosage form, similar in appearance and size to the sugar coated ‘100's and 1000's’ confectionary topping used on cakes, chocolate discs etc.

2. The individual solid dosage forms are blended in a scientifically formulated ratio to provide a targeted therapeutic dose or food supplement relative to the specific needs of the animal or human to deliver medication or correct low or inadequate nutrient levels in the diet.

3. The individual solid dosage forms (not powder) can be identified by the natural colour of the ingredients or colour coded by the addition of food colourings to make them distinct and easily recognized in the blend of the product formulated for a particular purpose, treatment or use.

4. The concept need not be restricted to an oral supplement or treatment, and may be used topically as a blended treatment for wounds etc, where the individual ingredients would not ordinarily be stable or maintain the activity, or would react chemically together, if they were mixed in the one formulation.

5. The concept of the examples is based on the separation of classes of nutrients, or even therapeutics, to prevent chemical interaction, as well as allowing these nutrients to be blended in varying ratios from bulk lots of the individual dosage forms to meet the specific needs of the animal.

For instance, trace-minerals, such as iron or copper react chemically with vitamins, such as Vitamin A, E and C to reduce their potency. In vitamin preparations, choline, classed as a B group vitamin, reacts chemically to destroy many other B group vitamins. However, it is not affected by trace-minerals, so it can be included in a separate trace-mineral mix to form solid doses. Even when in a dry powder form, destructive reactions can occur with those nutrients in close contact.

As previously described the dosage vehicles typically comprise a mass in the form of a mixture of dispersed portions of at least one active component in a substantially non-reactive separator material.

However, in some instances, the non-reactive separator material may itself include an active component. A case in point is where the substantially non-reactive separator material is calcium. If there is too much calcium in certain formulations these reduce the uptake of active components such as iron, zinc, magnesium and manganese. (It should also be noted that if there is too much zinc this can reduce the uptake of manganese).

In instances where calcium may reduce the uptake of such active components one can, for example, chose a leas reactive carrier such as, for example, DiCalcium Phosphate as a substitute for Calcium Carbonate.

As a general rule one should always select concentration and type of the separator material so that any active component which it might contain does not interfere with the uptake or therapeutic effect of the active component or components in the dosage vehicle.

The above describes only some embodiments of the present invention and modifications, obvious to those skilled in the art, can be made thereto without departing from the scope and spirit of the present invention.

1. A dosage system for delivery of predetermined quantities of predetermined selections of a therapeutically or nutritionally effective substance over predetermined time intervals into the body of an animal; said dosage system comprising:
(a) At a first location sorting a dosage vehicle according to an at least one primary characteristic;
(b) At said first location selecting and mixing at a first mixing time one or more of said dosage vehicles in accordance with a predetermined selection and predetermined quantity thereby to obtain a first dosage of a predetermined selection and predetermined quantity of said therapeutically or nutritionally effective substance specifically adapted for a predetermined target animal or target animal group;
(c) Transporting said first dosage to a second location;
(d) Selecting and mixing at a second mixing time said first dosage with a predetermined selection and predetermined quantity of feed material thereby to obtain a first feed mix;
(e) administering said first feed mix to said target animal or target animal group at a first predetermined administration time which is at or around said second mixing time;
(f) repeating steps (d) and (e) at predetermined repeat time intervals thereby to maintain a predetermined therapy profile in said target animal or target animal group over a predetermined therapy period.

2. The system of claim 1 further including attributing at least one sub-characteristic to an at least one dosage vehicle.

3. The system of claim 2 wherein said at least one sub-characteristic is such that it can be utilized to distinguish one dosage vehicle from another on the basis of said at least one primary characteristic.

4. The system of claim 2 wherein said dosage vehicle is sorted according to said at least one sub-characteristic.

5. The system of claim 2 including a second sub-characteristic.

6. The system of claim 5 wherein said second sub-characteristic is selected from odour, density, size, volume, diameter, length.

7. The system of claim 1 wherein said predetermined repeat time interval is 1 day.

8. The system of claim 1 wherein said predetermined repeat time interval is 0.5 day.

9. The system of claim 1 wherein said predetermined therapy period is at least six months.

10. The system of claim 1 wherein said predetermined therapy period is approximately 3 months.

11. The system of claim 1 wherein said predetermined therapy period is approximately 2 months.

12. The system of claim 1 wherein said predetermined therapy period is approximately 1 month.

13. The system of claim 1 wherein a second dosage is administered in a subsequent predetermined therapy period, thereby to tailor therapy for said animal as a function of time.

14. The system of claim 1 wherein said dosage vehicle is in the form of a predetermined quantity of a topical substance.

15. The system of claim 1 wherein said dosage vehicle is in the form of a predetermined quantity of a solid, ingestible substance.

16. The system of claim 15 wherein said solid, ingestible dosage is in the form of a pellet, prill, beadlet, bead, granule, tablet, caplet or flake.

17. The system of claim 1 wherein said predetermined target animal is selected from a dog, a pig, a cow or a fish.

18. The system of claim 1 wherein said predetermined target animal is a horse.

19. The system of claim 1 wherein said predetermined target animal is a human.

20. A method for delivery of predetermined quantities of predetermined selections of a therapeutically or nutritionally effective substance over predetermined time intervals into the body of an animal; said method comprising:
(a) At a first location sorting a dosage vehicle according to an at least one primary characteristic;
(b) At said first location selecting and mixing at a first mixing time one or more of said dosage vehicles in accordance with a predetermined selection and predetermined quantity thereby to obtain a first dosage of a predetermined selection and predetermined quantity of said therapeutically or nutritionally effective substance specifically adapted for a predetermined target animal or target animal group;
(c) Transporting said first dosage to a second location;
(d) Selecting and mixing at a second mixing time said first dosage with a predetermined selection and predetermined quantity of feed material thereby to obtain a first feed mix.
(e) administering said first feed mix to said target animal or target animal group at a first predetermined administration time which is at or around said second mixing time;
(f) repeating steps (d) and (e) at predetermined repeat time intervals thereby to maintain a predetermined therapy profile in said target animal or target animal group over a predetermined therapy period.

21. The method of claim 20 further including attributing at least one sub-characteristic to an at least one dosage vehicle.

22. The method of claim 21 wherein said at least one sub-characteristic is such that it can be utilized to distinguish one dosage vehicle from another on the basis of said at least one primary characteristic.

23. The method of claim 21 wherein said dosage vehicle is sorted according to said at least one sub-characteristic.

24. The method of claim 21 including a second sub-characteristic.

25. The method of claim 24 wherein said second sub-characteristic is selected from odour, density, size, volume, diameter, length.

26.-67. cancelled.
